(19) United States
(12) Patent Application Publication
(10) Pub. No.: US 2013/0012918 A1
(43) Pub. Date: Jan. 10, 2013

(54) STEADY GRIP OPHTHALMIC SURGICAL SYRINGE AND METHOD

(76) Inventor: Gary Foster, Fort Collins, CO (US)

(21) Appl. No.: 13/178,393

(22) Filed: Jul. 7, 2011

(51) Int. Cl. A61M 5/315; A61M 5/31

(52) U.S. Cl. 604/506; 604/227

(57) ABSTRACT

A disposable hypodermic syringe for stable one-handed dispensing of fluids and precision positioning comprises a hollow barrel for retaining fluid. The barrel has proximal end, a midpoint, and a distal end, and a proximal end. At least one forward barrel flange is fixedly formed around or connected to the barrel and positioned at a point between the midpoint and the distal end of the barrel.

A barrel and plunger with larger diameter and shorter length compared to conventional syringes of a given volume is disclosed.

A method of performing an injection using a syringe with a forward barrel flange is disclosed.

### BACKGROUND OF THE INVENTION

Many surgical procedures involve use of a syringe, for example to anesthetize the patient's eye. Different type of syringes have been developed which are useful for injecting and in some cases aspirating fluid in connection with surgical procedures.

Additionally, different types of needles and cannulas have been development which are useful for transmitting fluid between the barrel of the syringe an a desired location on or within a part of a patient's body, for example sometimes a syringe with a cannula is used to transmit fluid to or from an organ such as an eyeball.

Syringes are generally designed to be economical while providing the desired functions of injecting or aspirating.

### SUMMARY OF THE INVENTION

An embodiment of the invention may comprise a disposable hypodermic syringe for stable and precision positioning while dispensing a fluid with the action of a single hand comprising: a hollow barrel that retains fluid, the barrel having a proximal end, a midpoint, and a distal end, the proximal end adapted to receive a plunger; a tip capable of retaining a needle, the tip extending from the distal end of the barrel and having a conduit through the tip, the conduit providing fluid communication between an inside chamber of the barrel and the needle; a plunger having a thumb flange at a proximal end and a stopper at a distal end, the stopper configured to be in fluid-tight engagement with the inside chamber of the barrel; and at least one forward barrel flange fixedly connected to and disposed upon the barrel at a point distal to the midpoint of the barrel at a distance that accommodates simultaneous gripping of the distal end of the barrel and squeezing against the forward barrel flange without finger repositioning.

Another embodiment of the invention may comprise a method of dispensing a fluid with a syringe with the action of a single hand comprising: providing a syringe having a plunger and a hollow barrel, each having a distal end, a midpoint, and a proximal end, the barrel having at least one forward barrel flange fixedly connected to the barrel at a point between the midpoint of the barrel and the distal end of the barrel such that the forward barrel flange accommodates simultaneous gripping of the distal end of the barrel and squeezing against the forward barrel flange without finger repositioning; retaining a fluid within the hollow barrel between a stopper positioned on the distal end of the plunger and a hollow needle retained on the distal end of the barrel; gripping the syringe in the hand between a thumb flange disposed on the proximal end of the plunger and the forward barrel flange; and dispensing the fluid within the hollow barrel through the needle by depression of the plunger into the hollow barrel with pressure applied by the hand to decrease the distance between the thumb flange and the forward barrel flange

### BRIEF DESCRIPTION OF THE DRAWINGS

FIG. 1 illustrates a two-handed injection using a conventional syringe.

FIG. 2 illustrates attempting a one-handed injection using a conventional syringe.

FIG. 3A illustrates an embodiment of the steady grip ophthalmic surgical syringe gripped in a pencil grip and the plunger extended.

FIG. 3B illustrates an embodiment of the steady grip ophthalmic surgical syringe gripped in a pencil grip and the plunger depressed.

FIG. 4A illustrates an embodiment of the steady grip ophthalmic surgical syringe gripped in an alternative grip and the plunger extended.

FIG. 4B illustrates an embodiment of the steady grip ophthalmic surgical syringe gripped in an alternative grip and the plunger depressed.

FIG. 5 illustrates a view of an embodiment of the steady grip ophthalmic surgical syringe with self-aligning barrel flanges.

FIG. 6A illustrates a butterfly-shaped embodiment of extended barrel flanges.

FIG. 6B illustrates an embodiment of tapered barrel flanges.

FIG. 6C illustrates an embodiment of x-shaped extended barrel flanges.

FIG. 6D illustrates an embodiment of circular extended barrel flanges.

FIG. 7A illustrates a conventional syringe with rearwardly-disposed barrel flanges.

FIG. 7B illustrates an embodiment of the steady grip ophthalmic surgical syringe with forwardly-placed barrel flanges.

FIG. 8A illustrates a conventional syringe with rearwardly-disposed barrel flanges.

FIG. 8B illustrates an embodiment of the steady grip ophthalmic surgical syringe with forwardly-placed flanges and an enlarged diameter and a shortened barrel.

### DEFINITIONS

Shortened barrel as used herein is defined as a barrel accommodating a corresponding shortened plunger such that the distance between the thumb flange of the plunger and the distal end of the barrel at the point of a distal intersection with a conical portion connected to the tip conduit is within a designated short range when the plunger is extended to hold a designated volume of fluid. For example a range of about 7 cm to about 10 cm

Shortened plunger as used herein is defined as a plunger adapted to fit within a shortened barrel such that when the plunger is fully depressed within the shortened barrel, the distance which the distal end of the plunger extends distally from the distal end of the barrel is within a designated short range, for example, a range of about 0.3 cm to 3 cm.

Designated volume as used herein is defined as a volume of fluid which a syringe is intended to hold. For example, a syringe designated as a 3 ml syringe holds approximately 3 ml when the distal end of the plunger is positioned at a designated volume reference line. The plunger may be extended further whereby the volume within the barrel is greater than 3 ml or the plunger may be depressed whereby the volume within the barrel is less than 3 ml. Other typical designated volumes may include 5 ml, 10 ml, or any volume between 0.5 ml and 25 ml chosen as a designated volume.

Designated volume reference line as used herein is defined as the line of intersection between the distal end of the stopper and the proximal end of the barrel. In some embodiments, syringes may have a printed designated volume reference line, an unprinted designated volume reference line or they may be designed to provide mechanical feedback when the plunger is in the correct position to hold the designated volume.

A squat barrel as used herein is defined as a shortened barrel wherein the ratio of the barrel length to the barrel inside diameter is less than about 3.5.

### DETAILED DESCRIPTION OF THE EMBODIMENTS

Many ophthalmic surgical procedures involve a significant degree of precise fine motor movements. Procedures such as cataract surgery and Lasik surgery are often conducted with gross motor movements isolated and with the surgeon using only fine motor movements with the fingers. To keep the arms and hands steady during surgery, the surgeon may utilize elbow rests, wrist rests, or both.

Injections are also often performed in conjunction with the surgery, for example to anesthetize the patient's eye. Precision positioning of the injecting needle at the correct position and angle can be critical.

To use ordinary syringes in ophthalmic surgery has often required that the surgeon steady the position of the syringe with one hand while depressing the plunger of the syringe with the other hand.

Additionally, previous injection devices or systems intended to be performed one handed have been complicated and relatively expensive and could interfere with efficient and effective workflow needed for frequently performed ophthalmic procedures such as cataract and Lasik surgery.

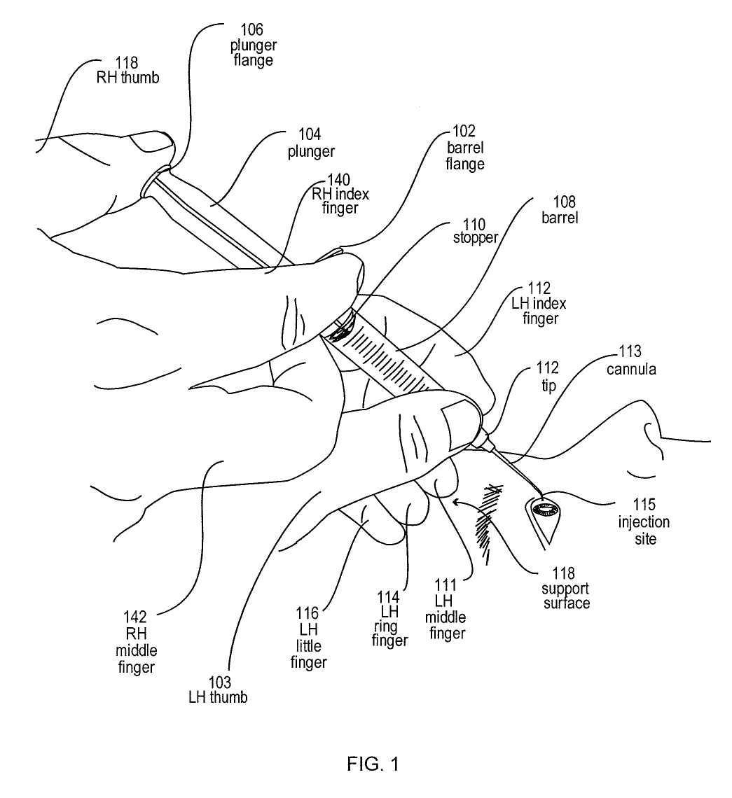

FIG. 1 shows a two-handed injection using a conventional syringe. In FIG. 1 the syringe barrel 108 is gripped in a pencil-grip at the distal end near the tip 112 between the left hand (LH) index finger 112, the LH middle finger 111 and the LH thumb 103. This pencil-grip enables the surgeon to precisely position and place the cannula 113 in the injection site 115. The middle 111, ring 114, and little 116 fingers of the hand gripping the syringe rest stably against the support surface 118, i.e. the patient's forehead. Thus the cannula 113, or alternatively a needle is securely gripped and positioned precisely ready for injection of a fluid.

To perform the injection, while maintaining precision placement of the cannula or needle, the surgeon may use the other hand, i.e. the right hand (RH). The surgeon grips the barrel flange 102 which is conventional at the proximal end of the barrel 108 with the pulps or pads of the RH index finger 140 and the RH middle finger and depresses the plunger 104 by pressing the plunger flange 106 with the RH thumb 118 until the desired amount of fluid is injected.

It should be noted that although FIG. 1 depicts the precision pencil grip and placement of the syringe being performed with the left hand and the depression of the plunger being performed with the right hand, these operations may be performed with the right hand positioning the needle or cannula and the left hand depressing the plunger according as desired by the surgeon. Another alternative may be to have an assistant perform the depression of the plunger using one or both of their hands.

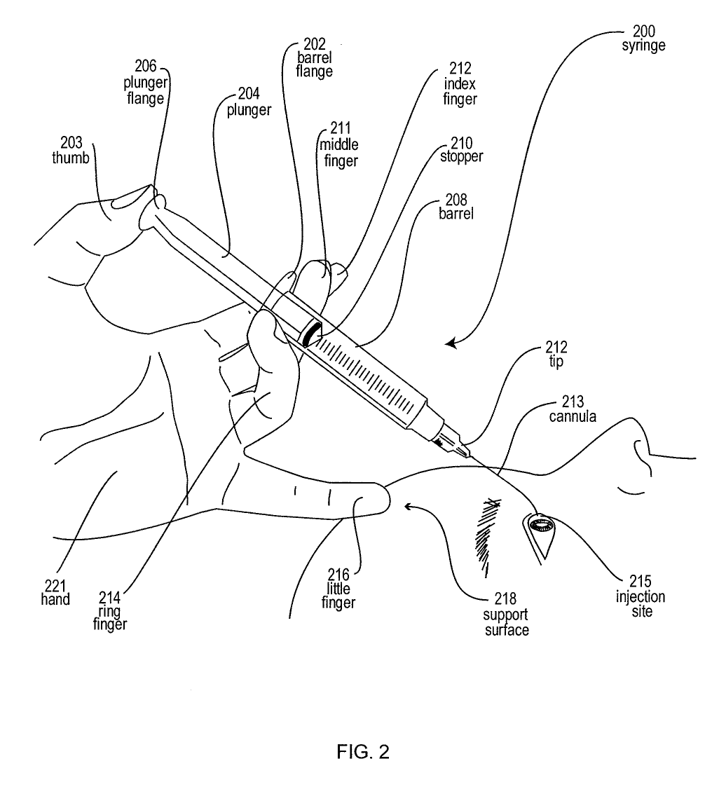

FIG. 2. illustrates a surgeon attempting to perform a one-handed injection which may be difficult depending on the dexterity and size of the surgeon's hands and the length of the syringe. This is because the needle or cannula 213 must enter the eye at a precise position and angle at the injection site 215. While endeavoring to maintain precise positioning of the needle or cannula 213, the surgeon must reach back with his or her thumb 203 and depress the plunger 204. This can be difficult, especially for surgeons with small hands. The little finger 216 may be extended to rest against the support surface 218, i.e. the patient's forehead. Although this may offer some stability, it may not be optimal due to the fact that the hand may wobble or move causing because there is only one point of stabilization. Depending on the viscosity of the fluid to be injected and the resistance of the opening, considerable force may need to be applied to depress the plunger 204 to perform the injection. This may require gross motor movement which interferes with the fine motor skills required to position and maintain the needle or cannula 213 in the proper position and angle at the injection site 215.

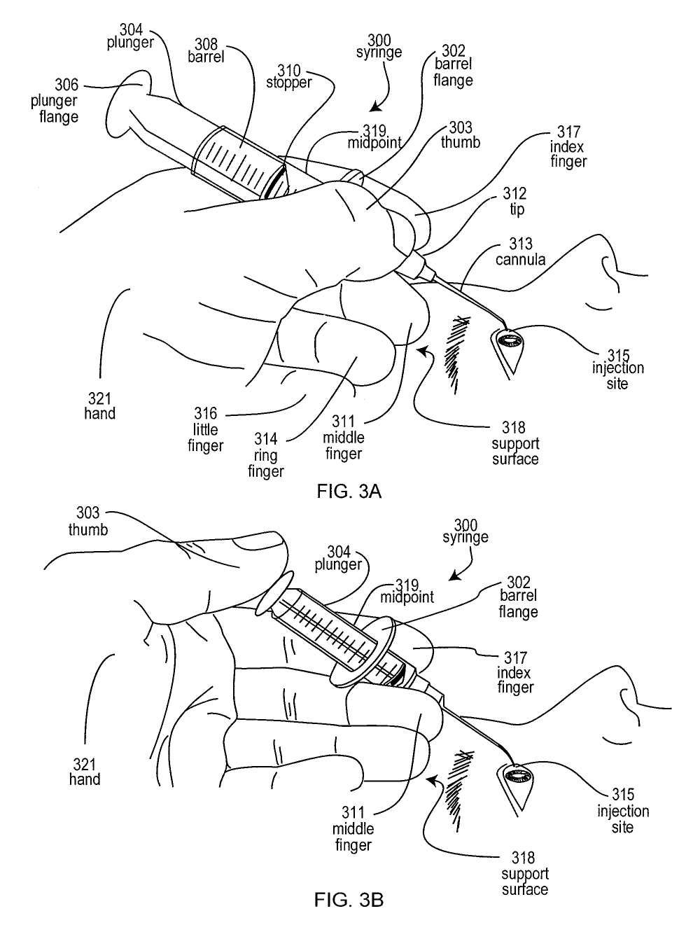

FIG. 3A. illustrates an embodiment of the ophthalmic syringe 300 and method of the invention. As shown in FIG. 3A, in this embodiment, the barrel 308 of the syringe is shorter in length then a conventional and the diameter of the syringe barrel 308 is larger than that a conventional syringe barrel, thus shortening the length while enabling the volume of fluid to be equivalent to that of conventional syringe.

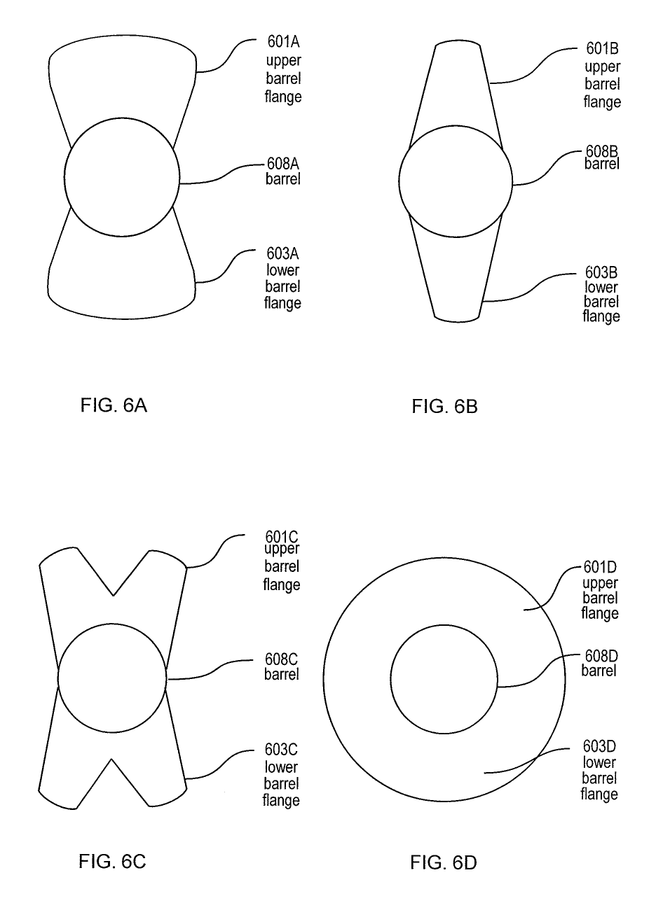

Also shown in FIG. 3A, is the forward or distal disposition of the barrel flange 302. In some embodiments, the barrel flange 302 is disposed between the midpoint 319 of the barrel 308 and the tip 312 of the barrel. A pencil grip may be comfortably used to grip the barrel 308 near the distal end or tip 312 using the thumb 303, the index finger 317, and the middle finger 311. By configuring the barrel flange 302 to be forwardly disposed, i.e. between the midpoint 319 and the tip 312, the fleshy part of the fingers, i.e. the pulps of index finger 317 and the middle finger 311, abuts the barrel flange while maintaining the pencil grip. This facilitates depression of the plunger as shown in FIG. 3B. It should be noted that while the barrel flange 302 depicted in FIGS. 3A and 3B are illustrated as oval or round barrel flanges such as illustrated in FIG. 6D and described below, the barrel flange may be of any desired size and shape as further described below with respect to FIGS. 6A-6D. In some embodiments, the flanges are shaped to maximize stability and contact with the pulps of the fingers when plunger is extended and when it is depressed.

FIG. 3B illustrates the surgeon performing the injection using syringe 300 by depressing the plunger 304 by simply moving the thumb 303 back to press against the plunger flange 306. The barrel is maintained stably in the correct position and angle as it is gripped between the index finger 317 and the middle finger 311. The hand remains stably resting upon the patient's forehead, i.e. the support surface. Because the lengths of the barrel 308 and the plunger 304 have been shortened, movement of the surgeon's thumb to the plunger flange 306 at the proximal end of the plunger 304 does not require larger motor movement nor opening of the hand. Requiring only the fine motor movement of rotating the thumb 303 back enhances the stability and ease of the injection.

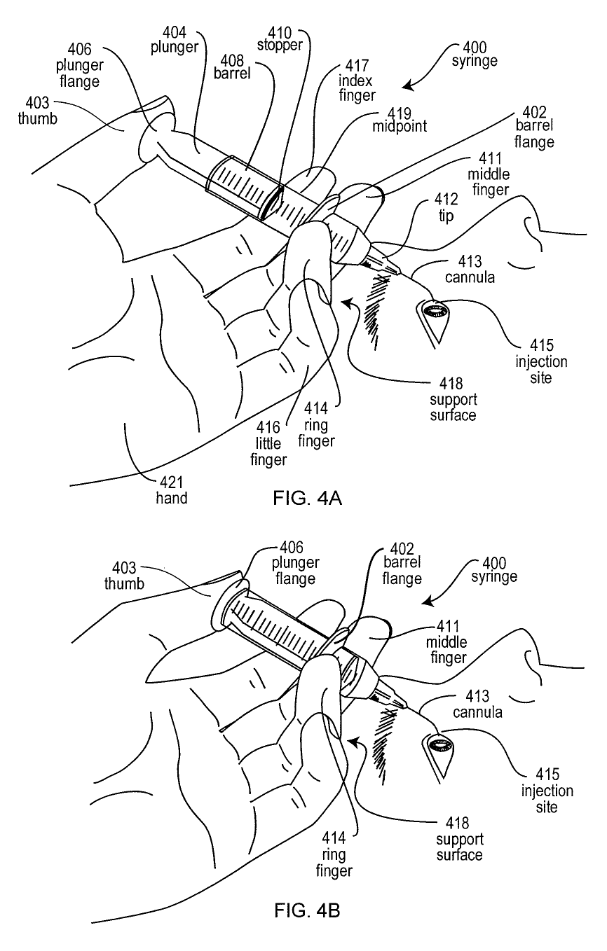

FIG. 4A shows another embodiment of the method wherein the syringe 400 is gripped near the distal end of the syringe barrel 408 between the middle finger 411 and the ring finger 414. The barrel flange 402 is forwardly disposed between the midpoint 419 of the barrel 408 and the tip 412. In some embodiments, the distance between the distal end of the barrel and the barrel flange 402 is about 1 to 2 finger widths which facilitates gripping of the barrel 408 near the distal end while pulps of the middle finger 411 and the ring finger 412 are touching the barrel flange 402 ready to perform the injection.

FIG. 4B shows the thumb 403 depressing the plunger flange 406 thereby injecting the desired fluid through the cannula 413 into the injection site 415. The embodiment of FIGS. 4A and 4B may be preferred by some surgeons by facilitating the back of hand 421 and fingers 414, 416, 411 to stably rest against the forehead of the patient, i.e. the support surface 418.

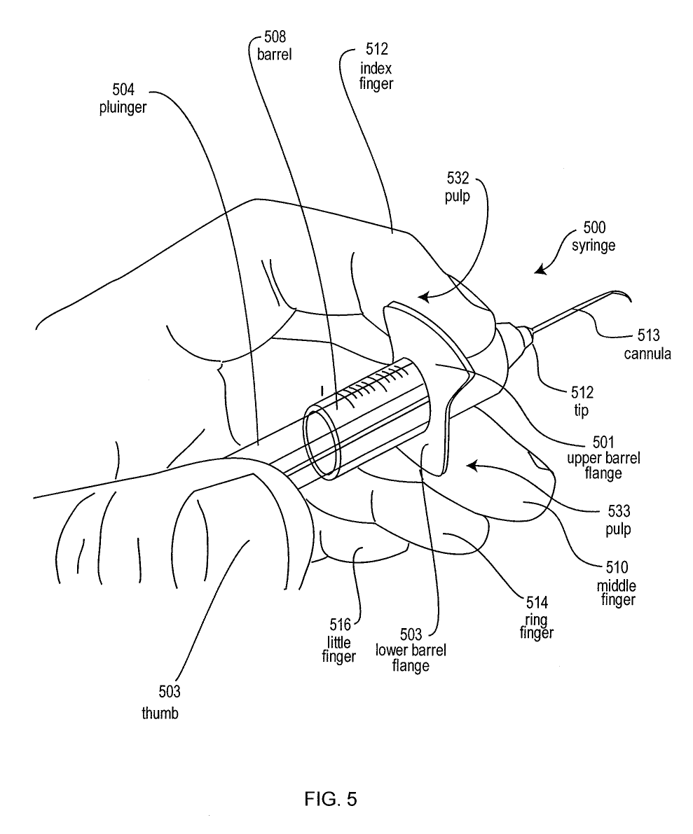

FIG. 5 is a view of syringe 500 from the proximal perspective showing an embodiment of forward barrel flange 502. The upper part of barrel flange 502 presses against pulp 532 of index finger 512 while the lower part of barrel flange 502 presses against pulp 533 of middle finger 511.

The barrel flange 502 may be formed as a single piece having a butterfly shape as showing in FIG. 5. Alternatively other sizes and shapes such as illustrated and described with respect to FIG. 6 may be used.

More than one flange may be used and adapted to align the syringe in a desired orientation. For example, in some embodiments a cannula 512 having a curved portion is attached to tip 512 and a locking mechanism such as a Luer lock facilitates the connection of the cannula 513 to the tip with a desired orientation, the barrel flange 502 or flanges may be adapted to conform and self-align comfortably to a desired finger position for the desired orientation. An upper part 501 of the barrel flange may be a different size or shape, or may be formed at a different angle than a lower part 503 of the barrel flange.

In other embodiments, the barrel flange 502 or flanges may be formed symmetrically so as to be easily utilized in either a right-handed or left-handed manner.

FIG. 6A shows a butterfly-shaped barrel flange 602A with an upper tab (also called a upper barrel flange) 601A and a lower tab (also called a lower barrel flange) 603A. The upper and lower tabs are so called to facilitate identification in the drawing. However, the lower tab 603A may be used in a manner where it is generally above the upper tab 601A and vice versa. In other words, the syringe may be gripped in any manner comfortable to the surgeon with the upper 601A and lower 603A tabs of the barrel flange 602A disposed in any manner that facilitates performing the injection.

FIG. 6B illustrates a tapered barrel flange 602B. The upper tab 601B and the lower tab 603B are tapered, although the shape and type of upper and lower flange parts need not be symmetrical nor symmetrically disposed with respect to each other. A tapered flange embodiment may comprise extending tabs that extend outwardly from the barrel and rest against pulps of two fingers when the two fingers grip the distal end of the barrel.

FIG. 6C is an X-wing shaped barrel flange 602C. An X-wing shaped barrel flange is one exemplary embodiment of a multi-tabbed flange having an upper barrel flange 601C with multiple tabs and a lower barrel flange 603C with multiple tabs. A multi-tabbed flange may comprise a plurality of extending tabs that extend outwardly from the barrel and rest against pulps of two fingers when the two fingers grip the distal end of the barrel. In the embodiment illustrated, a V-notch is shown in upper barrel flange 601C and lower barrel flange 603C. However any number of minimally extending portions, concavities, depressions, notches, indentions or the like may be used in a multi-tabbed flange. Likewise a multi-tabbed flange may comprise any number of extending tabs that extend outwardly from the barrel and rest against pulps of two fingers

FIG. 6D is a circular shaped barrel flange 602D wherein the upper part 601D and lower part 603D are configured to encircle barrel 608D.

In any of the embodiments such as those of FIGS. 6A, 6B, 6C, and 6D variations in the barrel flange may be used. For example any shape may be used with a first shaped upper part and a distinct second shaped lower part. The upper and lower parts of the barrel flanges may each connect to the barrel at the same distance between the midpoint and distal ends of the barrel or at different distances. Likewise one or both upper or lower parts of the flanges may connect at an angle adapted to accommodate stable comfortable contact between the barrel flange and the pulps of the fingers.

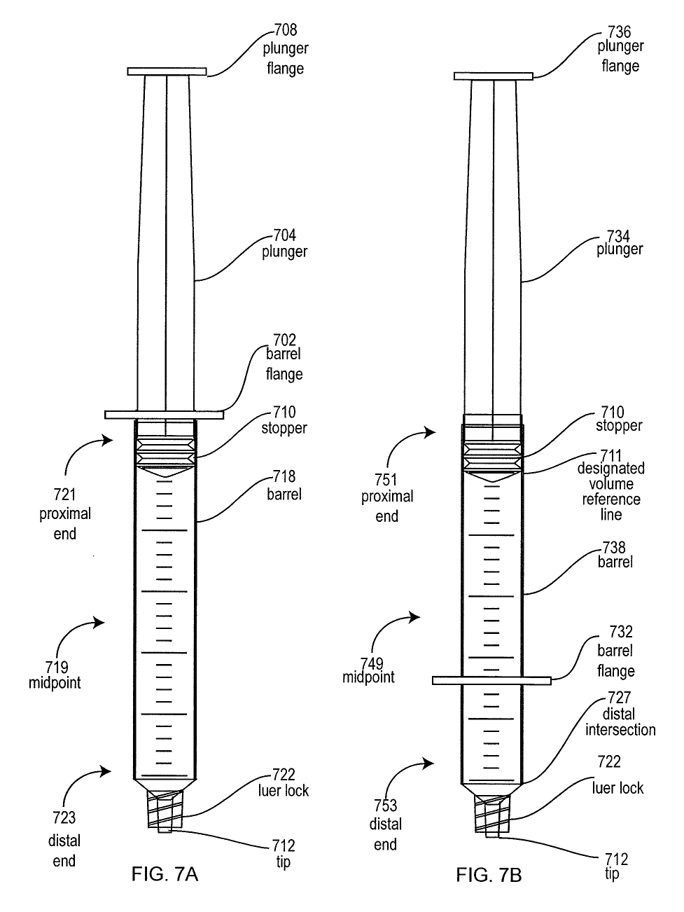

FIG. 7A a conventional syringe 700 and FIG. 7B illustrates an embodiment of a syringe 730 of the invention. The conventional syringe 700 includes a hollow barrel 708 for retaining fluid. The barrel 708 has a proximal end, a midpoint 719, and a distal end 723. The proximal end 721 of the barrel 708 is adapted to receive a plunger 704. The plunger 704 has a stopper 710 that is configured to be in fluid-tight engagement with the barrel 708. A tip 712 extends from the distal end 723 of the barrel 708 and is adapted to attach to a needle, a cannula, or in some other cases tubing. The tip 712 has a conduit through it that is in fluid communication with an inside chamber of the barrel 708. The tip 712 may be surrounded by a so called Luer lock 722 or any locking mechanism adapted to retain or position a needle or cannula.

In the conventional syringe 700, a barrel flange is disposed at or near the proximal end 721 of barrel 708. During injection, opposing pressure is applied to the plunger flange 706 and the barrel flange 702 to cause the plunger to push the stopper towards the distal end 723 of the barrel 708. The stopped is also moved while in fluid tight engagement of the barrel which causes fluid within the barrel to be forced through and out of tip 712 into a needle or cannula which may be attached.

Typical syringe volumes used in ophthalmic surgery may include 1 mL, 3 mL, or 5 mL. However, embodiments of the invention may be suitable for syringes having any desired volume for one handed ophthalmic injections.

FIG. 7B shows a syringe having many elements in common with the conventional syringe shown in 7A. However, the barrel flange 732 is a forward barrel flange fixedly connected to the barrel 738 and disposed at a position between the midpoint 749 of the barrel 738 and the distal end 753 of the barrel. This configuration enables a physician to grip the barrel 738 at the distal end 753 in a pencil grip or any desired grip which facilitates precision placement and maneuvering of the tip 742 which may have a needle or a cannula attached. While gripping the barrel 753 at the distal end 753, the barrel flange 732 is close to or even touching those fingers gripping the barrel so that such a grip may be maintained while the surgeon's thumb rotates back to depress the plunger flange 736 to perform the injection.

A designated volume reference line 711 is the line of intersection between the distal end of the stopper and the proximal end of the barrel as defined above in the definition section. In some embodiments, syringes may have a printed designated volume reference line, an unprinted designated volume reference line or they may be designed to provide mechanical feedback when the plunger is in the correct position to hold the designated volume. The designated volume of a barrel can be calculated as the volume of a cylinder which equals Pi (i.e. about 3.14159 . . . ) multiplied by the radius squared, multiplied by the length. The radius to be used is one half the inside diameter of the barrel and the length to be used is the length of the barrel from the distal intersection 727 (i.e. the intersection where the distal end of the barrel connects with a conical section that connects to the tip) and the designated volume reference line.

In some embodiments, a barrel 738 having a conventional or standard length and the diameter may be used for a typically used volume with an advantage of the invention being the forward barrel flange 732 fixedly formed around or connected to the barrel 738 so that pressure can be steadily applied during an ejection without the possibility of the barrel flange twisting, dislodging, or coming off.

For surgeons who can span a larger distance with their thumb, embodiments of the invention in which length and width are not altered from those of a conventional syringe may include the advantage of being able to use pre-existing or standard tooling in manufacturing.

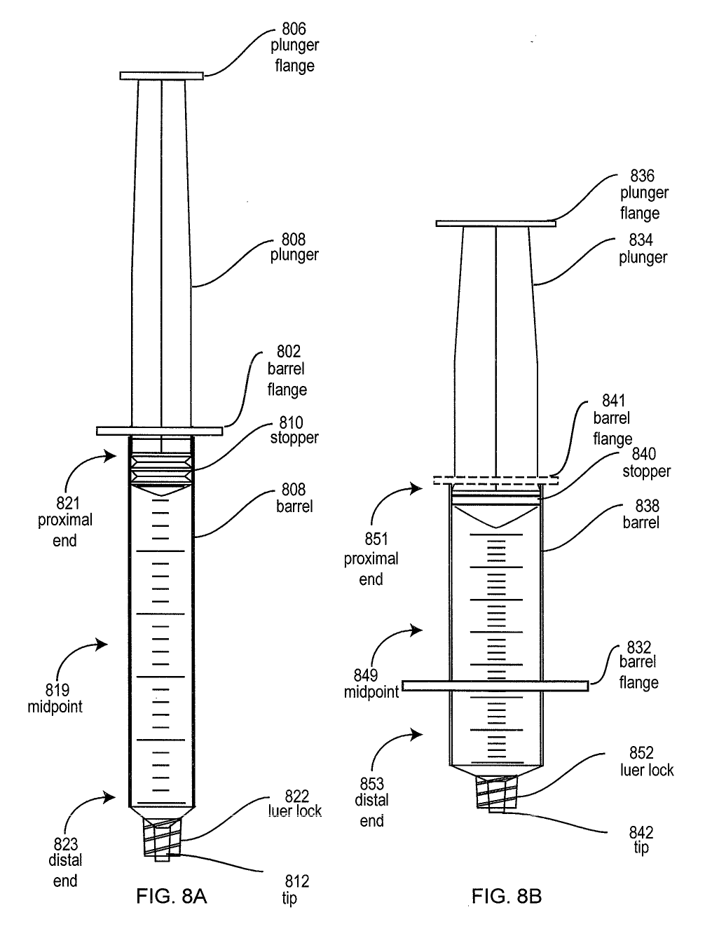

FIG. 8A illustrates a conventional syringe such as described above with respect to FIG. 7A. It is shown again for comparison with another embodiment of the invention as shown in FIG. 8B.

FIG. 8B is an embodiment of an ophthalmic surgical syringe 830 adapted for stable one-handed injections. As with other embodiments, the syringe 830 includes a forward barrel flange 832 fixedly formed or connected to barrel 838 at a position between the midpoint 844 and the distal end 853. As in other embodiments, a physician may grip the barrel 838 at the distal end 853 in a pencil grip or any desired grip which facilitates precision placement and maneuvering of the tip 842 which may have a needle or a cannula attached.

The length of the barrel 838 and the plunger 834 is shorter than that of a conventional syringe to reduce the distance of movement required of a surgeon's thumb to perform the injection. As previously describe, from a while gripping the barrel 858 at the distal end 853, the barrel flange 832 is close to or even touching those fingers gripping the barrel so that such a grip may be maintained while the surgeon's thumb rotates back to depress the plunger flange 836 to perform the injection.

The inside diameter of the barrel 838 and the corresponding diameter of the plunger 834 and stopper 840 have been enlarged to compensate for the decreased volume that would occur if the barrel length were shortened without a corresponding increase in the inside diameter of the barrel 838. Thus standard volumes may be maintained.

It may be desirable to increase the area and strength of the plunger flange 836 and the barrel flange 832 to facilitate the somewhat increased pressure required to depress the plunger 834 with a larger diameter.

With the embodiment of 8B as well as with any of the embodiments, in addition to the at least one forward barrel flange 832, a second, proximal barrel flange 841 may optionally be disposed at the proximal end of the barrel 851 at a standard location. The at least one proximal barrel flange 841 may be oriented in line with the forward barrel flange 832 or may be oriented at an angle different than that of the forward barrel flange 832, for example, the proximal barrel flange 841 may be oriented at 90 degrees to the forward barrel flange 831. This allows the operator to use either set of flanges to inject fluid depending on the application. In addition, the 90 degree orientation allows the proximal barrel flange 841 to rest against the hand to steady the syringe while using the forward barrel flange 832 to inject a fluid.

The various embodiments shown and illustrated provide an ophthalmic surgical syringe and corresponding methods well-suited for precision placement and stability while performing a one-handed injection.

What is claimed is:

1. A disposable hypodermic syringe for stable and precision positioning while dispensing a fluid with the action of a single hand comprising:
a hollow barrel that retains fluid, said barrel having a proximal end, a midpoint, and a distal end, said proximal end adapted to receive a plunger;
a tip capable of retaining a needle, said tip extending from said distal end of said barrel and having a conduit through said tip, said conduit providing fluid communication between an inside chamber of said barrel and said needle;
a plunger having a thumb flange at a proximal end and a stopper at a distal end, said stopper configured to be in fluid-tight engagement with said inside chamber of said barrel; and
at least one forward barrel flange fixedly connected to and disposed upon said barrel at a point distal to said midpoint of said barrel at a distance that accommodates simultaneous gripping of said distal end of said barrel and squeezing against said forward barrel flange without finger repositioning.

2. The syringe of claim 1, wherein said barrel is a shortened barrel and said plunger is a shortened plunger such that a maximum distance between said thumb flange of said plunger and said distal end of said barrel at a point of intersection of said barrel and said tip conduit is in a range of about 5 cm to about 10 cm when said plunger is extended to enclose a designated full volume of fluid, and wherein said shortened barrel is a squat barrel such that a ratio of a length of said inside chamber of said barrel to a diameter of said inside chamber of said barrel is less than 3.5.

3. The syringe of claim 2 wherein said diameter of said barrel is such that said barrel is configured to dispense designated full volume of about 3 ml.

4. The syringe of claim 2 wherein said diameter of said barrel is such that said barrel is configured to dispense designated full volume of about 5 ml.

5. The syringe of claim 2 wherein said diameter of said barrel is such that said barrel is configured to dispense designated full volume of about 10 ml.

6. The syringe of claim 1 wherein said forward barrel flange is butterfly-shaped further comprising:
a pair of concave portions that do not extend substantially beyond a diameter of said barrel; and,
a pair of convex tabs that extend outwardly from said barrel and provide a surface to accommodate finger pulp pressure.

7. The syringe of claim 1 wherein said forward barrel flange is a multi-tabbed flange further comprising:
three or more concave portions that do not extend substantially beyond a diameter of said barrel; and,
three or more tabs that extend outwardly from said barrel and provide a surface to accommodate finger pulp pressure.

8. The syringe of claim 1 wherein said forward barrel flange is a circular flange that extends outwardly from said barrel to accommodate finger pulp pressure.

9. The syringe of claim 1 wherein said forward barrel flange is an axially angled flange comprising at least one tab that extends outwardly at a non-perpendicular angle from an axis of said barrel to accommodate finger pulp pressure.

10. The syringe of claim 1 further comprising at least one proximal barrel flange fixedly connected to and disposed upon said barrel at said distal end of said barrel.

11. The syringe of claim 10 wherein the proximal barrel flange extends in a radially rotated angle relative to said forward barrel flange.

12. A method of dispensing a fluid with a syringe with the action of a single hand comprising:
providing a syringe having a plunger and a hollow barrel, each having a distal end, a midpoint, and a proximal end, said barrel having at least one forward barrel flange fixedly connected to said barrel at a point between said midpoint of said barrel and said distal end of said barrel such that said forward barrel flange accommodates simultaneous gripping of said distal end of said barrel and squeezing against said forward barrel flange without finger repositioning;
retaining a fluid within said hollow barrel between a stopper positioned on said distal end of said plunger and a hollow needle retained on said distal end of said barrel;
gripping said syringe in said hand between a thumb flange disposed on said proximal end of said plunger and said forward barrel flange; and
dispensing said fluid within said hollow barrel through said needle by depression of said plunger into said hollow barrel with pressure applied by said hand to decrease the distance between said thumb flange and said forward barrel flange.
